(19) United States
(12) Patent Application Publication
(10) Pub. No.: US 2015/0057595 A1
(43) Pub. Date: Feb. 26, 2015

(54) PRESSURE-BASED FLOW RATE MEASUREMENT FOR OCULAR IMPLANTS

(71) Applicant: ALCON RESEARCH, LTD., Fort Worth, TX (US)

(72) Inventors: Nicholas Max Gunn, Newport Beach, CA (US); Andrew David Johnson, Tustin, CA (US)

(73) Assignee: ALCON RESEARCH, LTD., Fort Worth, TX (US)

(21) Appl. No.: 13/975,322

(22) Filed: Aug. 24, 2013

(51) Int. Cl. A61F 9/007

(52) U.S. Cl. CPC A61F 9/00781
USPC 604/9

(57) ABSTRACT

Described herein is an IOP control device for implantation in an eye of a patient, comprising a drainage tube, a pressure-driven flow system, and a flow rate measurement system. The drainage tube includes a drainage lumen in fluid communication with an anterior chamber of the eye. The flow system is in fluid communication with the drainage lumen, and is configured to control the flow of fluid through the drainage tube. The flow rate measurement system is disposed distal to the flow system, and comprises a flow tube including a known hydraulic resistance to flow, a proximal pressure sensor disposed at the proximal end of the flow tube, and a distal pressure sensor disposed at the distal end of the flow tube. The flow tube includes a proximal end, a distal end, and a lumen extending therebetween that is configured to be in fluid communication with the drainage lumen.

### BACKGROUND

The present disclosure relates generally to valves and associated systems and methods. In some instances, embodiments of the present disclosure are configured to be part of an intraocular pressure (IOP) control system for use in ophthalmic treatments.

Glaucoma, a group of eye diseases affecting the retina and optic nerve, is one of the leading causes of blindness worldwide. Most forms of glaucoma result when the IOP increases to pressures above normal for prolonged periods of time. IOP can increase due to high resistance to the drainage of the aqueous humor relative to its production. Left untreated, an elevated IOP causes irreversible damage to the optic nerve and retinal fibers resulting in a progressive, permanent loss of vision.

FIG. 1 is a diagram of the front portion of an eye that helps to explain the processes of glaucoma. In FIG. 1, representations of the lens 110, cornea 120, iris 130, ciliary body 140, trabecular meshwork 150, and Schlemm's canal 160 are pictured. Anatomically, the anterior segment of the eye includes the structures that cause elevated IOP which may lead to glaucoma. Aqueous humor fluid is produced by the ciliary body 140 that lies beneath the iris 130 and adjacent to the lens 110 in the anterior segment of the eye. This aqueous humor washes over the lens 110 and iris 130 and flows to the drainage system located in the angle of the anterior chamber 170. The angle of the anterior chamber 170, which extends circumferentially around the eye, contains structures that allow the aqueous humor to drain. The trabecular meshwork 150 is commonly implicated in glaucoma. The trabecular meshwork 150 extends circumferentially around the anterior chamber. The trabecular meshwork 150 may act as a filter, limiting the outflow of aqueous humor and providing a back pressure that directly relates to IOP. Schlemm's canal 160 is located beyond the trabecular meshwork 150. Schlemm's canal 160 is fluidically coupled to collector channels (not shown) allowing aqueous humor to flow out of the anterior chamber. The two arrows in the anterior segment of FIG. 1 show the flow of aqueous humor from the ciliary bodies 140, over the lens 110, over the iris 130, through the trabecular meshwork 150, and into Schlemm's canal 160 and its collector channels.

One method of treating glaucoma includes implanting a drainage device in a patient's eye. The drainage device allows fluid to flow from the anterior chamber of the eye to a drainage site, relieving pressure in the eye and thus lowering IOP. In order to provide desired treatments to patients, it may be important to regulate the flow of aqueous humor through the drainage device. Some flow regulation devices may be able to measure the pressure within the anterior chamber as well as the pressure at the drainage site of the implant and use these pressure measurements to influence the flow of aqueous humor through the device. However, the actual rate of aqueous flow through these pressure-based flow regulating devices may be unknown. Therefore, it may be desirable to provide flow regulation devices capable of monitoring the actual rate of aqueous flow through the device.

The system and methods disclosed herein overcome one or more of the deficiencies of the prior art.

### SUMMARY

In one exemplary aspect, this disclosure is directed to an IOP control device for implantation in an eye of a patient, comprising a drainage tube sized for implantation into the eye of a patient, a pressure-driven flow system in fluid communication with the drainage tube, and a flow rate measurement system. In one aspect, the drainage tube includes a drainage lumen in fluid communication with an anterior chamber of the eye. In one aspect, the pressure-driven flow system is in fluid communication with the drainage lumen, and is configured to control the flow of fluid through the drainage tube. In one aspect, the flow rate measurement system is disposed distal to the flow system, and comprises a flow tube, a proximal pressure sensor, and a distal pressure sensor. In one aspect, the flow tube includes a proximal end, a distal end, and a lumen extending therebetween that is configured to be in fluid communication with the drainage lumen. In one aspect, the proximal pressure sensor is disposed proximal of the proximal end, and the distal pressure sensor is disposed distal of the distal end. The flow tube includes a known hydraulic resistance to flow.

In one exemplary aspect, the present disclosure is directed to an IOP control device for implantation in an eye of a patient, comprising a drainage tube, a flow system, a flow rate measurement system, and a controller. The drainage tube is sized for implantation into the eye of a patient. In one aspect, the drainage tube includes a drainage lumen in fluid communication with an anterior chamber of the eye. In one aspect, the flow rate measurement system is disposed distal to the flow system, which is in fluid communication with the drainage lumen. In one aspect, the flow rate measurement system comprises a flow tube, a proximal pressure sensor, and a distal pressure sensor. In one aspect, the flow tube includes a proximal end, a distal end, and a lumen extending therebetween that is configured to be in fluid communication with the drainage lumen. In one aspect, the proximal pressure sensor is disposed at the proximal end, and the distal pressure sensor is disposed at the distal end. The flow tube includes a known hydraulic resistance to flow. In one aspect, the controller is configured to receive data from the proximal pressure sensor and data from the distal pressure sensor, and is configured to determine flow rate based on the data from the proximal pressure sensor, the data from the distal pressure sensor, and pre-stored information relating to the hydraulic resistance to flow.

In another exemplary embodiment, the present disclosure is directed to a method of monitoring flow rate of fluid from an anterior chamber of an eye through an implantable device. The method comprises directing fluid through a drainage tube containing a flow measurement system and a flow system configured to control the flow of fluid from the anterior chamber through the drainage tube to a drainage site. In one aspect, the flow measurement system comprises a flow tube having a known hydraulic resistance to flow, a proximal pressure sensor disposed between the flow system and the flow tube, and a distal pressure sensor disposed between the flow tube and the drainage site. The method further comprises measuring a first pressure proximal to the flow tube using the proximal pressure sensor, measuring a second pressure distal to the flow tube using the distal pressure sensor, and calculating the flow rate through the drainage tube using the known hydraulic resistance to flow of the flow tube, the first pressure, and the second pressure.

It is to be understood that both the foregoing general description and the following detailed description are exemplary and explanatory in nature and are intended to provide an understanding of the present disclosure without limiting the scope of the present disclosure. In that regard, additional aspects, features, and advantages of the present disclosure will be apparent to one skilled in the art from the following detailed description.

### BRIEF DESCRIPTION OF THE DRAWINGS

The accompanying drawings illustrate embodiments of the devices and methods disclosed herein and together with the description, serve to explain the principles of the present disclosure.

FIG. 1 is a diagram of the front portion of an eye.

FIG. 2 is an illustration of an exemplary flow-regulating system disposed in the eye in accordance with one embodiment of the present disclosure.

FIG. 3 is a block diagram of an exemplary IOP control system according to the principles of the present disclosure.

FIG. 4 is a stylized diagram of an exemplary IOP control system including an exemplary flow rate measurement system according to the principles of the present disclosure disposed within an eye.

FIG. 5 is a schematic diagram of an exemplary IOP control system including an exemplary flow rate measurement system according to the principles of the present disclosure.

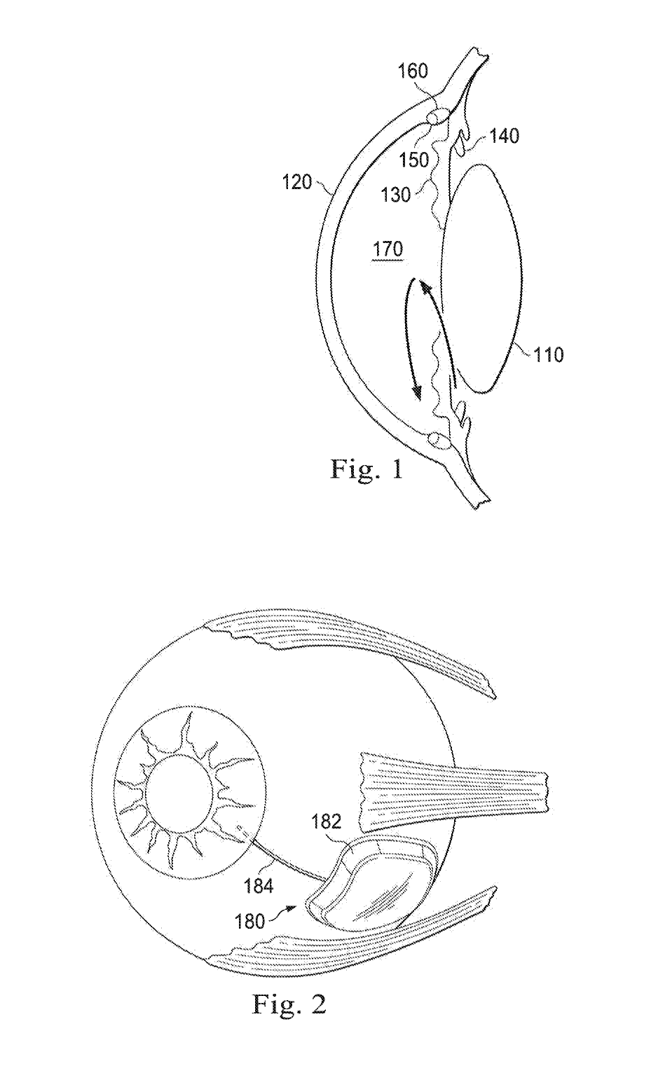

### DETAILED DESCRIPTION

For the purposes of promoting an understanding of the principles of the present disclosure, reference will now be made to the embodiments illustrated in the drawings, and specific language will be used to describe the same. It will nevertheless be understood that no limitation of the scope of the disclosure is intended. Any alterations and further modifications to the described devices, instruments, methods, and any further application of the principles of the present disclosure are fully contemplated as would normally occur to one skilled in the art to which the disclosure relates. In particular, it is fully contemplated that the features, components, and/or steps described with respect to one embodiment may be combined with the features, components, and/or steps described with respect to other embodiments of the present disclosure. For the sake of brevity, however, the numerous iterations of these combinations will not be described separately. For simplicity, in some instances the same reference numbers are used throughout the drawings to refer to the same or like parts.

The present disclosure relates generally to a flow rate measurement system to monitor the rate of fluid flow though a flow passageway or tube. In some instances, embodiments of the present disclosure are configured to be part of an IOP control system. In some instances, embodiments of the present disclosure are configured to be disposed within or carried onboard an implantable glaucoma drainage device. In some instances, embodiments of the present disclosure are configured to be used in the operation of pressure-driven membrane valves within an implantable glaucoma drainage device. The flow rate measurement system disclosed herein calculates the flow rate through a flow passageway including at least a portion having a known hydraulic resistance by measuring pressures upstream and downstream of this portion.

Thus, an IOP control system utilizing the flow rate measurement system disclosed herein enables the real-time measurement of the aqueous flow rate through an implanted drainage device, which may facilitate better monitoring and treatment of the disease (e.g., glaucoma) than IOP control systems that operate based on pressure measurements alone. The flow rate measurement systems disclosed herein may guide treatment regimens in response to changes in the aqueous humor outflow rate. For example, in at least one embodiment, knowledge of the real-time flow rate of aqueous humor through the drainage device enables the user and/or the IOP control system to change the pumping schedule of the drainage device in order to better regulate aqueous outflow from the anterior chamber through the drainage device. Those of skill in the art will realize that the flow rate measurement system disclosed herein may be utilized in alternative applications requiring flow rate measurement through a flow passageway.

FIG. 2 shows an exemplary implantable system 180 disposed on an eye to treat an ocular condition according to one exemplary aspect of the present disclosure. The implantable system 180 includes a body referred to herein as a plate 182 and a drainage tube 184 that extends from the plate 182. The plate 182 is arranged to carry various components of an IOP control system, and may include a flow rate measurement device, a valve, a pump, transducers or sensors, a processing system and memory, drug delivery components, a power source, or other components that may be used to either control the implantable system 180 or otherwise treat ocular conditions.

When implanted, the plate 182 may be located in the subconjunctival pocket between the conjunctiva and sclera. It may be generally located on an ocular quadrant commonly used for conventional glaucoma drainage devices with plates; that is, it may be centered such that it is located between the neighboring ocular muscles that define the ocular quadrant chosen for implantation. In the pictured embodiment, the plate 182 is configured to fit at least partially within the subconjunctival space and is sized for example within a range between about 15 mm×10 mm to about 30 mm×15 mm. In some embodiments, the plate 182 has a thickness less than about 2 mm thick. For example, in one embodiment, the plate has a thickness of about 1 mm thick. The plate 182 may be curved to approximate the radius of the eye globe. In some embodiments, the plate 182 is rigid and preformed with a curvature suitable to substantially conform to the globe or it may be flexible to conform to the globe. The above dimensions and arrangement are exemplary only, and other sizes and arrangements are contemplated.

The drainage tube 184 is sized to extend from the plate 182 to the anterior chamber of the eye. The drainage tube 184 bridges the anterior chamber and the plate 182 in the subconjunctival pocket to provide an auxiliary flow path for aqueous humor, bypassing the flow-resistive conventional pathway through the trabecular meshwork and shunting aqueous humor directly to a drainage site. In the example shown, the drainage tube 184 is a single tube having a single lumen. Other embodiments include a plurality of drainage tubes or a plurality of lumens cooperating together to permit fluid to flow through the implantable system 180. Aqueous humor may drain through the drainage tube from the anterior chamber to and out of the plate 182 to alleviate elevated intraocular pressure conditions.

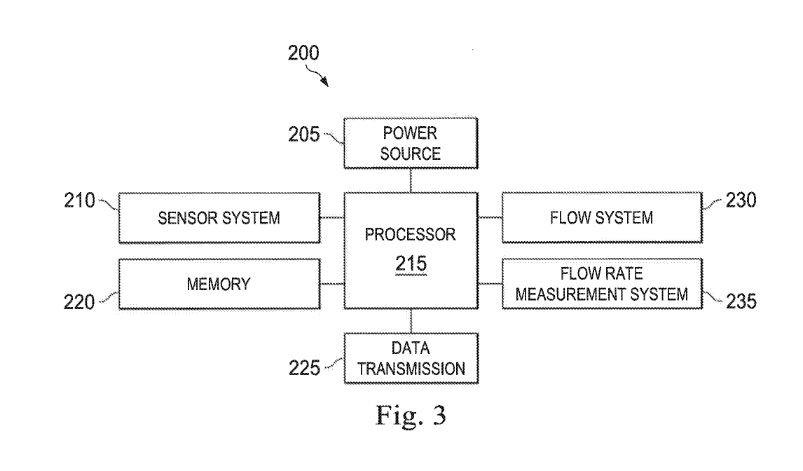

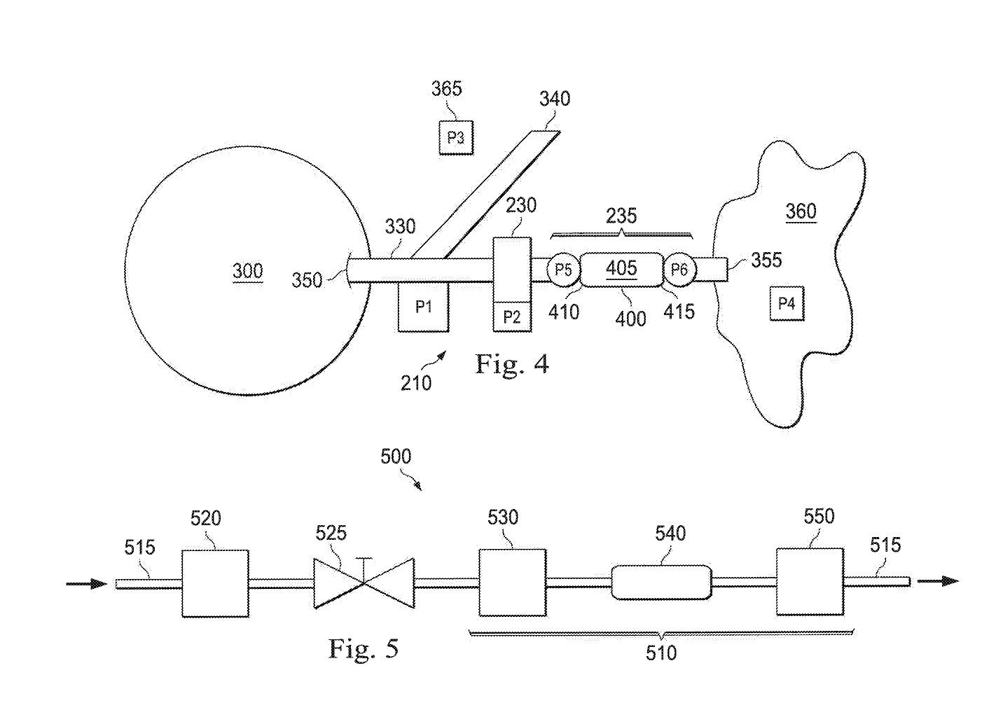

FIG. 3 is a block diagram of an exemplary IOP control system 200, which is at least partially implantable in an eye of a patient for the treatment of glaucoma or other conditions. In particular, a portion or all of the IOP control system 200 may be carried on or may form a part of the implantable system 180. In one embodiment, it is carried on the plate 182. The IOP control system 200 is configured in a manner that provides IOP pressure control. In FIG. 3, the IOP control system 200 includes a power source 205, an IOP sensor system 210, a processor 215, a memory 220, a data transmission module 225, a flow system 230, and a flow rate measurement system 235. Many of the elements of the IOP control system 200 are described below. The IOP sensor system 210 and the flow rate measurement system 235 are described in further detail below with reference to FIG. 4.

The power source 205, which provides power to the system 200, is typically a rechargeable battery, such as a lithium ion or lithium polymer battery, although other types of batteries may be employed. In other embodiments, any other type of power cell is appropriate for the power source 205. The power source can be recharged via inductive coupling such as an RFID link or other type of electromagnetic coupling.

The processor 215 is typically an integrated circuit with power, input, and output pins capable of performing logic functions. For example, the processor 215 may perform logic functions based on inputs from the IOP sensor system 210 and/or the flow rate measurement system 235 to determine the current IOP of the eye, the flow rate through the implantable system 180 (shown in FIG. 2), and the operating status of the IOP control system 200. The processor 215 performs mathematical calculations utilizing sensed pressure data from and known characteristics of the flow rate measurement system 235 to calculate the flow rate of aqueous fluid through the flow system 230. In some embodiments, the processor 215 performs mathematical calculations utilizing sensed pressure data from the IOP sensor system 210 to calculate the IOP. In some embodiments, the processor 215 controls the supply of power from the power source 205 to the flow system 230, the IOP sensor system 210, and/or the flow rate measurement system 235. In various embodiments, the processor 215 may be a targeted device controller or a microprocessor configured to control more than one component of the device or a combination thereof.

The processor 215 may include one or more programmable processor units running programmable code instructions for modulating flow through the flow system 230, among other functions. The processor 215 may be integrated within a computer and/or other processor-based devices suitable for a variety of ocular applications. In some embodiments, the processor 215 regulates flow through the flow system 230 based on a programmable regimen, which may be carried by such a device and/or the memory 220. In some instances, the programmable regimen includes specific command signals to the flow system 230 based on different input data from the IOP sensor system 210 and/or the flow rate measurement system 235. In some embodiments, the processor 215 can receive input data from the IOP sensor system 210 and/or the flow rate measurement system 235 via wireless or wired mechanisms. The processor 215 may use such input data to generate control signals to control or direct the operation of the flow system 230 and thereby affect the rate of fluid flow from the anterior chamber. In some embodiments, the processor 215 is offboard the plate 182 and is in direct wireless communication with the IOP sensor system 210, the flow rate measurement system 235, and/or the flow system 230, and can receive data from and send commands to these component parts of the IOP control system 200.

The memory 220, which is typically a semiconductor memory such as RAM, FRAM, or flash memory, interfaces with the processor 215. As such, the processor 215 can write to and read from the memory 220, and perform other common functions associated with managing semiconductor memory. In this manner, a series of flow rate calculations, command algorithms, and/or pressure readings can be stored in the memory 220.

The processor 215 and/or the memory 220 may also include software containing one or more algorithms or programmable regimens defining one or more functions or relationships between command signals and input data (e.g., input data received from the flow rate measurement system 235). The algorithm may dictate command signals to the flow system 230 depending on the received input data or mathematical derivatives thereof.

The data transmission module 225 may employ any of a number of different types of data transmission. For example, in various embodiments, the data transmission module 225 may be an active device such as a radio or a passive device with an antenna capable of wireless transmission. Alternatively, the data transmission module 225 may be activated to communicate an elevated IOP condition or a real-time flow rate to a secondary device such as a PDA, cell phone, computer, wrist watch, custom device for this purpose, remote accessible data storage site (e.g. an internet server, email server, text message server), or other electronic device or service. Additionally, the data transmission module 225 may be utilized to program or reprogram the device with alternate treatment/control schedules.

FIG. 4 is a stylized diagram of the IOP sensor system 210 disposed about a representation of an eye, the flow system 230, a drainage tube 330, a divider 340, and the exemplary flow rate measurement system 235. The drainage tube 330 may be the same drainage tube 184 discussed with respect to FIG. 2 and drains aqueous humor from the anterior chamber 300 of the eye. The flow system 230 and the flow rate measurement system 235 are disposed along, and may form a part of, the drainage tube 330 between the tube end in the anterior chamber 300 and the drainage site 360. The flow system 230 controls the flow of aqueous humor through the tube 330 and may comprise one or more valves, one or more pumps, or a combination of valves and pumps, or other flow devices for regulating or otherwise affecting flow. The flow measurement system 235 monitors the flow rate from the anterior chamber 300 through the drainage tube 330 to the drainage site 360.

In FIG. 4, the exemplary IOP sensor system 210 (shown in FIG. 3) includes four pressure sensors, P1, P2, P3, and P4. The pressure sensor P1 is located in or is in fluidic communication with an anterior chamber 300, the pressure sensor P2 is located to measure intermediate pressures found within the flow system 230, the pressure sensor P3 is located remotely from P1 and P2 in manner to measure atmospheric pressure, and the pressure sensor P4 is located at the drainage site 360 and is arranged to measure drainage pressure, such as a bleb pressure. In some embodiments, the IOP sensor system includes three pressure sensors, corresponding to the sensors P1, P3, and P4 shown in FIG. 4. In particular, the IOP control system 200 may lack a pressure sensor located to measure intermediate pressures within the flow system 230 (e.g., the pressure sensor P2). In some embodiments, the IOP control system 200 may include any number of pressure sensors or may lack the IOP sensor system 210 altogether.

In some embodiments, the pressure sensor P1 is located in a lumen or tube that is in fluid communication with the anterior chamber, such as the drainage tube 330. In the embodiment shown, the pressure sensor P1 measures the pressure in the tube 330 upstream from the flow system 230 and downstream from the anterior chamber 300. In this manner, pressure sensor P1 measures the pressure in the anterior chamber 300 because the expected measurement discrepancy between the true anterior chamber pressure and that measured by P1 when located in a tube downstream of the anterior chamber (even when located between the sclera and the conjunctiva) is very minimal.

In some embodiments, the system includes barriers that separate the sensors P1, P2, P3, and P4. These barriers may be elements of the system itself. For example, in FIG. 4, the pressure sensor P3 is physically separated from the pressure sensor P4 by the divider 340. The divider 340 is a physical structure that separates the wet drainage site 360 of P4 from a dry site 365 of P3. In one example, the barrier separating the anterior chamber pressure sensor P1 and the drainage site pressure sensor P4 is the flow system 230. Some embodiments may lack a divider 340.

Generally, IOP is a gauge pressure reading—the difference between the absolute pressure in the eye (as measured by P1) and atmospheric pressure (as measured by P3). In one embodiment of the present disclosure, pressure readings are taken by the pressure sensors P1 and P3 simultaneously or nearly simultaneously over time so that the actual IOP can be calculated (as P1-P3 or P1−f(P3), where f(P3) indicates a function of P3). The pressure readings of P1 and P3 can be stored in memory 220 by the processor 215. They can later be read from memory so that actual IOP over time can be interpreted by a physician.

The pressure sensor P4 may be located in a pocket at the drainage site 360, such as a bleb, that generally contains aqueous humor or in communication with such a pocket, via a tube for example, and is in the drainage site 360. The drainage site 360 may be, by way of non-limiting example, in a subconjunctival space, a suprachoroidal space, a subscleral space, a supraciliary space, Schlemm's canal, a collector channel, an episcleral vein, and a uveo-scleral pathway, among other locations in the eye. The difference between the readings taken by the pressure sensor P1 and the pressure sensor P4 (P1-P4) provides an indication of the pressure differential between the anterior chamber 300 and the drainage site 360. In one embodiment, this pressure differential dictates the rate of aqueous humor flow from the anterior chamber 300 to the drainage site 360.

In FIG. 4, the exemplary flow measurement system 235 (shown in FIG. 3) includes a flow tube 400, a proximal pressure sensor P5, and a distal pressure sensor P6. The flow tube 400 is shaped and configured to allow the passage of fluid through a lumen 405 extending from a proximal end 410 of the flow tube 400 to a distal end 415 of the flow tube 400. The flow tube 400 is a fluid conduit having a known resistance to flow (i.e., a relatively constant hydraulic resistance). The resistance to flow of the flow tube 400 is based on the physical characteristics of the flow tube 400, including, by way of non-limiting example, length, diameter, material type, roughness of the conduit, lubricity, and other physical characteristics. The flow tube 400 may be shaped in any of a variety of shapes, including, by way of non-limiting example, a cylindrical tube, a square channel, or a combination thereof. Various IOP control systems 200 may include different flow tubes 400 having distinct resistances to flow based on the desired application. In some embodiments, the flow tube 400 of the flow measurement system 235 may be fabricated as an integral part of the drainage tube 330. In other embodiments, the flow tube 400 may be a distinct fluid conduit or length of tubing in fluidic communication with the drainage tube 330. The flow tube 400 may be constructed of any of a variety of materials having high stiffness and small tolerances, such as, by way of non-limiting example, glass channel or tube, sapphire channel or tube, silicon channel or tube, plastic channel or tube, or a silicone tube.

The pressure sensors P5 and P6 flank the entrance and exit of the flow tube 400. In particular, the pressure sensor P5 is located proximal to the flow tube 400, and the pressure sensor P6 is located distal to the flow tube 400. The flow tube 400 and the pressure sensors P5 and P6 are arranged and configured relative to the drainage tube 330 to allow aqueous humor from the flow system 230 to flow from the drainage tube 330, past the proximal pressure sensor P5, through the lumen 405 of the flow tube 400 from the proximal end 410 to the distal end 415, and past the distal pressure sensor P6 toward the drainage site 360. Aqueous humor exits the flow tube 400 at the distal end 415 to pass the pressure sensor P6 for release at the drainage site 360. In the pictured embodiment, the pressure sensors P5 and P6 are disposed immediately adjacent the proximal end 410 and the distal end 415, respectively, of the flow tube 400. Thus, the proximal pressure sensor P5 measures the pressure in the flow path within the drainage tube 330 immediately before the proximal end 410 of the flow tube 400, and the distal pressure sensor P6 measures the pressure in the flow path within the drainage tube 330 immediately after the distal end 415 of the flow tube 400. In other embodiments, any one or both of the pressure sensors P5 and P6 are located a distance apart from the flow tube 400. In some embodiments, any one or both of the pressure sensors P5 and P6 are located within the flow tube 400 near or at the proximal end 410 and the distal end 415, respectively, of the flow tube 400.

The pressure sensors P1, P2, P3, P4, P5, and P6 can be any type of pressure sensors suitable for implantation in the eye. They each may be the same type of pressure sensor, or they may be different types of pressure sensors. In other embodiments, the flow rate measurement system 235 may include more than two pressure sensors. For example, in one embodiment, the flow rate measurement system 235 may include two proximal pressure sensors P5 and/or two distal pressure sensors P6 disposed within the drainage tube 330 at different positions relative to the flow tube 400. In other embodiments, a differential pressure sensor may be used to measure the difference across lumen 405, representing the difference between P5 and P6.

The flow system 230 is configured to control the flow of drainage fluid through the drainage tube 330, and thereby control pressure in the eye, including the IOP. The flow system 230 is configured to selectively allow or block the flow of aqueous humor flowing from the anterior chamber 300 through the drainage tube 330 and the flow tube 400 to the drainage site 360. The flow system 230 may include any number and combination of flow control structures such as, by way of non-limiting example, valves, pumps, and/or check valves before entering the drainage site. For example, in some embodiments, the flow system 230 comprises a series of valves. In other embodiments, the flow system 230 comprises a pump. In some embodiments, the flow system 230 comprises a combination of valves and pumps. Some examples of valves that comprise the flow system 230 include membrane valves, check valves, reed valves, pressure relief valves, and other types of valves. It is worth noting that for biocompatibility, the devices disclosed herein may be coated or encapsulated in a material such as polypropylene, silicon, Parylene, or other materials.

A desired pressure differential can be maintained by monitoring and controlling the flow rate through the flow system 230. For example, when IOP is high and/or the flow rate through the drainage tube 330 is low, the flow system 230 may operate to permit increased flow through the drainage tube 330, and when IOP is low and/or the flow rate through the drainage tube 330 is elevated, the flow system 230 may operate to decrease the flow through the drainage tube 330. Likewise, some embodiments of the flow rate measurement system 235 are configured to monitor the flow rate of drainage fluid to the drainage site 360 or bleb, and thereby enable the user and/or processor 215 to control the bleb pressure to maintain a desired fluid flow to the bleb and thereby decrease fibrosis and increase absorption efficiency. To accomplish this, the flow rate measurement system 235 may convey pressure measurements sensed by the pressure sensors P5 and P6 to the processor 215, and the processor 215 may utilize that data to select a control algorithm and/or send particular command signals to the flow system 230 to appropriately adjust the flow rate through the drainage tube 330. In some embodiments, the flow system 230 may also be responsive to instructions from the processor 215 based on input data received from the pressure sensors P5 and P6, the known resistance of the flow tube 400, and the calculated flow rate. The pressure readings of P5 and P6 as well as the calculated flow rates over time may be stored in memory 220 by the processor 215. They can later be read from memory so that actual flow rates over time can be interpreted by a physician. With the flow rate data and IOP values, it is then possible to determine if the bleb is still viable or if some form of surgical intervention is required to improve outflow performance. In some embodiments, the flow system 230 may be responsive to instructions from the processor 215 based on input data received from the pressure sensors P1, P2, P3, and/or P4, and the calculated IOP. In some embodiments, the flow system 230 may be responsive to instructions from the processor 215 based on a pre-programmed treatment protocol or input data received from the IOP sensor system as well as the flow rate measurement system 235.

The IOP control system 200 is configured to adjust the flow through the flow system 230 based on measured pressure values or derivatives from the pressure sensors P5 and P6 of the flow rate measurement system 235. After the implantable portion of the IOP control system 200 is implanted within the eye of a patient (e.g., a drainage device including the drainage tube 330, the flow system 230, and the flow rate measurement system 235), the flow rate measurement system 235 may communicate real time measured pressures from the proximal pressure sensor P5 and the distal pressure sensor P6 to the processor 215 (i.e., at a time T1). The processor 215 calculates the real time flow rate through the drainage tube 330 based on known mathematical principles of flow rate based on the pressure drop across a fluid conduit having a known resistance to flow. For example, in at least one embodiment, the processor 215 calculates the flow rate through the flow tube 400 and the drainage tube 330 by employing the equation Q=ΔP/R, where Q is the flow rate, ΔP is the pressure drop across the flow tube 400 (e.g., P5-P6), and R is the known hydraulic resistance of the flow tube 400.

If the processor 215 determines that the flow rate is not within a desired range, the IOP control system 200 may adjust the flow system 230 to increase or decrease drainage flow through the drainage tube 330 to effect a flow rate change to a desired flow rate (and may thereby effect a pressure change to the desired IOP). To do this, the processor 215 operates the flow system 230 with the power source 205 to change the flow rate through the flow system 230. After adjusting flow through the flow system 230, the flow rate measurement system 235 can again measure and communicate real time measured pressures from the proximal pressure sensor P5 and the distal pressure sensor P6 to the processor 215 (i.e., at a time T2 later than the time T1). The processor 215 can calculate this second flow rate using the same mathematical principles described above to reevaluate the flow through the drainage tube 330 and appropriately adjust the flow through the flow system 230 based on the calculated flow rate. In some embodiments, the processor 215 also evaluates pressure readings from the IOP sensor system 210 and calculations thereof before adjusting the operation of the flow system 230.

FIG. 5 is a schematic diagram of an exemplary IOP control system 500 including an exemplary flow rate measurement system 510 according to the principles of the present disclosure. The IOP control system 500 may be substantially identical to the IOP control system 200 discussed with respect to FIGS. 3 and 4 except for the differences described herein. The flow rate measurement system 510 may be substantially identical to the flow rate measurement system 235 discussed with respect to FIGS. 3 and 4 except for the differences described herein.

FIG. 5 illustrates various components of the IOP control system 500, which comprises a flow rate measurement system 510, a drainage tube 515, an anterior chamber pressure sensor 520, a flow system 525, a proximal pressure sensor 530, a flow tube 540, and a distal pressure sensor 550. The flow system 525 may be the same flow system 235 described above in relation to FIGS. 2-4. The flow system 525 is configured to selectively allow, block, or otherwise regulate the flow of aqueous humor flowing through the drainage tube 515 and the flow tube 510 to the drainage site 360 (shown in FIG. 4). In the illustrated embodiment, the flow rate measurement system 510 comprises the proximal pressure sensor 530, the flow tube 540, and the distal pressure sensor 550. In some embodiments, the flow tube 540 may be shaped and configured as a continuous, integral part of the drainage tube 515. In some embodiments, the flow tube 540 may have the same cross-sectional dimensions as the drainage tube 515 (e.g., without limitation, the same inner diameter, the same outer diameter, and the same wall width). In the illustrated embodiment, the proximal pressure sensor 530 and the distal pressure sensor 550 are disposed a distance apart from the flow tube 540. In other embodiments, as described above in relation to FIG. 4, these pressure sensors may be disposed immediately adjacent to the flow tube 540. In operation, the flow rate measurement system 510 may function in substantially the same manner as that described above in relation to the flow rate measurement system 235 (in FIG. 4).

The devices, systems, and methods described herein achieve IOP control using a relatively small and inexpensive flow rate measurement system disposed on an implantable drainage device. In some embodiments, the exemplary IOP control system disclosed herein uses data from the flow rate measurement system as well as an IOP sensor system to affect drainage flow, thereby taking into account intraocular pressure and/or bleb pressures to affect drainage flow through the drainage tube. In other embodiments, the IOP control system disclosed herein may lack an IOP sensor system and operate to affect flow through the drainage tube based mainly on pressure data from the flow rate measurement system.

In particular, the flow rate measurement system disclosed herein enables IOP control systems to better monitor the disease state (e.g., glaucoma progression) and to more effectively guide treatment regimens (e.g., pumping schedules) in response to changes in aqueous humor outflow rate through the implanted drainage device. Use of the flow rate measurement system disclosed herein in the IOP control system enables the use of better control algorithms that are able to take into account the actual, real time flow rate of aqueous humor through the drainage device as opposed to being based on raw pressure measurements alone.

In addition, the simple design of the flow measurement system may result in a thinner implant that will likely be more comfortable for the patient. In addition, because the flow tube of the flow measurement system may be fabricated as an integral part of the drainage tube, the overall manufacturing process may be simplified and costs may be reduced.

Embodiments in accordance with the present disclosure may be used in a variety of applications to monitor flow and/or pressure. For example, but not by way of limitation, embodiments of the present disclosure may be utilized to monitor flow rates in a flow control system as part of a dialysis system, a process control system, and/or a drug delivery system. Some embodiments of the present disclosure may be utilized to monitor flow rates in a variety of fluid flow implants such as, but not by way of limitation, the urinary tract, the brain (e.g., to regulate intracranial pressure), and the circulatory/renal system (e.g., as part of a dialysis system).

Persons of ordinary skill in the art will appreciate that the embodiments encompassed by the present disclosure are not limited to the particular exemplary embodiments described above. In that regard, although illustrative embodiments have been shown and described, a wide range of modification, change, and substitution is contemplated in the foregoing disclosure. It is understood that such variations may be made to the foregoing without departing from the scope of the present disclosure. Accordingly, it is appropriate that the appended claims be construed broadly and in a manner consistent with the present disclosure.

We claim:

1. An IOP control device for implantation in an eye of a patient, comprising:
a drainage tube sized for implantation into the eye of a patient and including a drainage lumen in fluid communication with an anterior chamber of the eye;
a pressure-driven flow system in fluid communication with the drainage lumen, the flow system being configured to control the flow of fluid through the drainage tube; and
a flow rate measurement system disposed distal to the flow system, the flow rate measurement system comprising:
a flow tube including a proximal end, a distal end, and a lumen extending therebetween, the lumen configured to be in fluid communication with the drainage lumen;
a proximal pressure sensor disposed proximal of the proximal end; and
a distal pressure sensor disposed distal of the distal end,
wherein the flow tube includes a known hydraulic resistance to flow.

2. The IOP control device of claim 1, wherein the flow tube is an integral part of the drainage tube.

3. The IOP control device of claim 1, wherein the flow tube includes a flow tube inner diameter that is equivalent to a drainage tube inner diameter.

4. The IOP control device of claim 1, wherein the proximal pressure sensor is disposed within the drainage tube adjacent to the proximal end of the flow tube.

5. The IOP control device of claim 1, wherein the distal pressure sensor is disposed within the drainage tube adjacent to the distal end of the flow tube.

6. The IOP control device of claim 1, wherein the proximal pressure sensor is disposed within the drainage tube a distance apart from the proximal end of the flow tube, and the distal pressure sensor is disposed within the drainage tube a distance apart from the distal end of the flow tube.

7. The IOP control device of claim 1, wherein the proximal pressure sensor and the distal pressure sensor are disposed within the flow tube.

8. The IOP control device of claim 1, further including an IOP sensor system comprising at least one pressure sensor.

9. The IOP control device of claim 8, wherein the IOP sensor system comprises an anterior chamber pressure sensor disposed within the drainage tube proximal to the flow system and an atmospheric pressure sensor.

10. The IOP control device of claim 1, further including a processor configured to calculate a flow rate through the drainage tube based on pressure data received from the flow measurement system.

11. The IOP control device of claim 10, wherein the processor is configured to regulate flow through the drainage tube by adjusting the flow system in response to the flow rate.

12. The IOP control device of claim 11, wherein the processor is configured to regulate flow through the drainage tube based on a programmable regimen.

13. An IOP control device for implantation in an eye of a patient, comprising:
a drainage tube sized for implantation into the eye of a patient and including a drainage lumen in fluid communication with an anterior chamber of the eye;
a flow system in fluid communication with the drainage lumen;
a flow rate measurement system disposed distal to the flow system, the flow rate measurement system comprising:
a flow tube including a proximal end, a distal end, and a lumen extending therebetween, the lumen configured to be in fluid communication with the drainage lumen;
a proximal pressure sensor disposed at the proximal end; and
a distal pressure sensor disposed at the distal end,
wherein the flow tube includes a known hydraulic resistance to flow; and
a controller configured to receive data from the proximal pressure sensor and data from the distal pressure sensor, the controller being configured to determine flow rate based on the data from the proximal pressure sensor, the data from the distal pressure sensor, and pre-stored information relating to the hydraulic resistance to flow.

14. The IOP control device of claim 13, further including a pressure-driven flow system in fluid communication with the drainage lumen, the flow system being configured to control the flow of fluid through the drainage tube.

15. The IOP control device of claim 13, wherein the flow tube is an integral part of the drainage tube.

16. A method of monitoring flow rate of fluid from an anterior chamber of an eye through an implantable device, comprising:
directing fluid through a drainage tube containing a flow measurement system and a flow system configured to control the flow of fluid from the anterior chamber through the drainage tube to a drainage site, wherein the flow measurement system comprises a flow tube having a known hydraulic resistance to flow, a proximal pressure sensor disposed between the flow system and the flow tube, and a distal pressure sensor disposed between the flow tube and the drainage site;
measuring a first pressure proximal to the flow tube using the proximal pressure sensor;
measuring a second pressure distal to the flow tube using the distal pressure sensor;
calculating the flow rate through the drainage tube using the known hydraulic resistance to flow of the flow tube, the first pressure, and the second pressure.

17. The method of claim 16, wherein calculating the flow rate through the drainage tube comprises dividing the difference between the first pressure and the second pressure by the known hydraulic resistance to flow through the flow tube.

18. The method of claim 16, further comprising communicating the first pressure and the second pressure to a processor to calculate the flow rate through the drainage tube.

19. The method of claim 18, further comprising modifying the amount of flow through the drainage tube by adjusting the flow system based on the flow rate.

20. The method of claim 19, wherein modifying the amount of flow through the drainage tube by adjusting the flow system based on the flow rate comprises adjusting the flow system in response to a programmable regimen.
